(19) United States
(12) Plant Patent Application Publication
(10) Pub. No.: US 2017/0290247 P1
(43) Pub. Date: Oct. 5, 2017

(54) Coleus plant named 'Sakcol019'

(50) ;

(71) Applicant: Sakata Seed Corporation, Yokohama (JP)

(72) Inventors: Masanori Ikeno, Kakegawa (JP); Rei Takahashi, Kakegawa (JP)

(73) Assignee: Sakata Seed Corporation, Yokohama (JP)

(21) Appl. No.: 14/999,095

(22) Filed: Mar. 30, 2016

(51) Int. Cl. A01H 5/12

(52) U.S. Cl. CPC A01H 5/12
USPC PLT/373

(57) ABSTRACT

A coleus plant particularly distinguished by large leaves and yellow-green leaves with green margins, is disclosed.

Genus and species: Solenostemon sp.

Variety denomination: ‘SAKCOL019’.

### BACKGROUND OF THE NEW PLANT

The present invention comprises a new and distinct variety of coleus, botanically known as Solenostemon sp., and hereinafter referred to by the variety name ‘SAKCOL019’.

The new plant was first asexually propagated in 2013 in Japan and has been asexually reproduced by vegetative cuttings for almost three years in Japan. The present invention has been found to retain its distinctive characteristics through successive asexual propagations by vegetative cuttings.

‘SAKCOL019’ has not been made publicly available or sold more than one year prior to the filing date of this application.

### SUMMARY OF THE NEW PLANT

The following are the most outstanding and distinguishing characteristics of the new variety when grown under normal horticultural practices in Salinas, Calif.

1. Large leaves; and

2. Yellow-green leaves with green margins.

### DESCRIPTION OF THE PHOTOGRAPHS

This new coleus plant is illustrated by the accompanying photographs which show the overall plant habit, and foliage of the plant. The colors are as true as can be reasonably obtained by conventional photographic procedures.

FIG. 1 shows the whole plant, including habit and foliage.

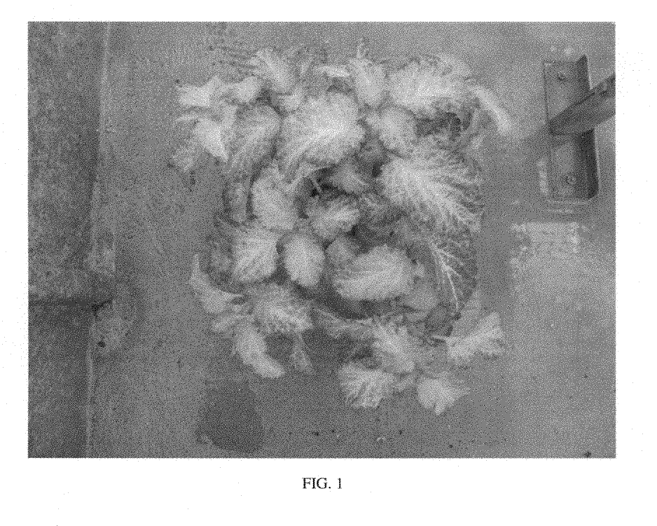

FIG. 2 shows a close-up of the foliage.

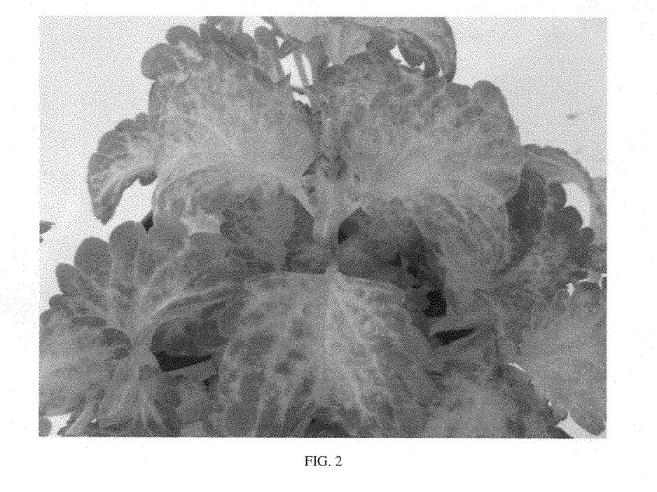

### DESCRIPTION OF THE NEW VARIETY

The following detailed descriptions set forth the distinctive characteristics of ‘SAKCOL019’. The data which defines these characteristics were collected from asexual reproductions.
Classification:
   Family.—Lamiaceae.
   Botanical name.—Solenostemon sp.
   Common name.—Coleus.
   Denomination.—‘SAKCOL019’.
Plant:
   Type.—Annual.
   Habit.—Upright, compact.
   Propagation type.—Vegetative cuttings.
Leaves:
   Arrangement.—Opposite/Sub-opposite.
   Color.—Yellow-green with green margins.
   Margin.—Crenate.
Disease or insect resistance: No disease or insect resistance observed.

We claim:

1. A new and distinct variety of coleus plant named ‘SAKCOL019’ as illustrated and described herein.
